(19) United States
(12) Patent Application Publication
(10) Pub. No.: US 2020/0129398 A1
(43) Pub. Date: Apr. 30, 2020

(54) Hair Cleansing and Conditioning Composition

(71) Applicant: Lonza Ltd., Visp (CH)

(72) Inventors: Louise Reay, Bebington (GB); Victor Low, Jersey City, NJ (US); Jordan Todorov Petkov, Stockport (GB); Elias Pambou, Blackley (GB)

(21) Appl. No.: 16/665,799

(22) Filed: Oct. 28, 2019

Related U.S. Application Data
(60) Provisional application No. 62/752,103, filed on Oct. 29, 2018.

(51) Int. Cl. A61K 8/41; A61K 8/42; A61K 8/37; A61K 8/36; A61K 8/34; A61Q 5/02; A61Q 5/12

(52) U.S. Cl. CPC A61K 8/416; A61K 8/42; A61K 8/375; A61K 8/361; A61K 2800/596; A61Q 5/02; A61Q 5/12; A61K 2800/30; A61K 8/342

(57) ABSTRACT

The present invention provides a hair cleansing conditioner composition containing a) 0.01 w/w %-20 w/w % of a cationic surfactant; b) 0.01 w/w %-20 w/w % of a polyglyceryl ester; and c) 0.01 w/w %-15 w/w %, of at least one fatty alcohol. A method of cleansing hair is also described.

### RELATED APPLICATIONS

The present application is based on and claims priority to U.S. Provisional Patent application Ser. No. 62/752,103, filed on Oct. 29, 2018, which is incorporated herein by reference.

### FIELD OF INVENTION

The disclosure relates to a hair cleansing and conditioning composition which both conditions and cleans hair.

### BACKGROUND OF THE INVENTION

Conventional hair cleansers usually contain anionic surfactants, which serve to clean the hair. Anionic surfactants are often desired and are typically necessary so that sebum and other soiling can be removed from the hair surface and the scalp. However, the use of anionic surfactants to cleanse hair will also result in a removal of lipids and proteins from the hair and/or the scalp during the cleaning process. This results in the necessity for a subsequent treatment with nourishing agents for the hair to revitalize the hair the hair is necessary. This can result in damage to the hair. In addition to daily cleaning, the structure of hair can also be damaged by environmental influences (such as intense solar radiation), mechanical stresses (such as harsh combing under the heat of a hair dryer), and physical or chemical treatments (such as dyeing, shaping or smoothing the hair). The consequences are often split-ends, hair breakage and/or a lack of hair shine. Hair damaged in this manner is often difficult to style, often becomes entangled, and when entangled, can be difficult to untangle

Hair conditioners or hair conditioning treatments are applied to hair for the intended purpose to replace the oils washed out of the hair by shampoos, to provide nourishing agents for regenerating the hair and repair any damage to the hair caused by cleaning the hair. There is a need in the art, to provide a more mild hair cleanser without removing oils from the hair while simultaneously nourishing the hair; leaving hair cleaned, shiny, damage free and manageable.

The present disclosure provides an answer to that need of having a hair cleansing and conditioning composition that does not have the above-mentioned disadvantages. In particular, a hair cleansing and conditioning composition is described herein provide cleansing of the hair, but does so with a composition which is essentially free of any anionic (sulphate) surfactants. Without the anionic surfactants, the composition provides and ensures a particularly mild cleansing. At the same time, hair of any type, but in particular brittle, damaged, dull, dry, thin and/or lank hair, should be effectively nourished, so that it has a softer feel and has greater volume, elasticity and shine following the treatment with the hair cleansing and conditioning composition.

### SUMMARY OF THE INVENTION

The present invention provides a hair cleansing and conditioning composition that contains a cationic surfactant: a polyglyceryl ester, and at least one fatty alcohol. It has been discovered that the combination of these ingredients provides for a composition that can be used to both cleanse and condition hair, with limited use of anionic surfactants.

In one aspect, the present invention provides hair cleansing and conditioning composition containing a) 0.001 w/w %-20 w/w % of a cationic surfactant; b) 0.01 w/w %-20 w/w % of a polyglyceryl ester surfactant or emulsifier; and c) 0.01 w/w %-15 w/w %, of at least one fatty alcohol.

In one embodiment, the cationic surfactant contains a quaternary ammonium compound; wherein least one quaternary ammonium compound comprises a cetyltrimethylammonium compound, behenyltrimethylammonium compound, cetylpyridinium compound, tetramethylammonium compound, tetraethylammonium compound, octyltrimethylammonium compound, dodecyltrimethylammonium compound, hexadecyltrimethylammonium compound, octyldimethylbenzylammonium compound, decyldimethylbenzylammonium compound, stearyldimethylbenzylammonium compound, didodecyldimethylammonium compound, dioctadecyldimethylammonium compound, tallowtrimethylammonium compound, dihydrogenated tallow dimethyl ammonium compound, cocotrimethylammonium compound, or PEG-2-oleammonium compound or a mixture of two or more of these compounds. Each of the compounds may have, wherein each of the above compounds has a halide or a sulfate anion. Suitable halides include, for example chloride and bromide. Suitable sulfates include, for example, metholsulfate.

In another embodiment, the amidoamine may be a stearamido-propyldimethyl amine, stearamidopropyldiethyl amine, stearamidoethyldiethyl amine, stearamidoethyldimethyl amine, palmitamidopropyldimethyl amine, palmitamidopropyl-diethyl amine, palmitamidoethyldiethyl amine, palmitamidoethyldimethyl amine, behenamidopropyldimethyl amine, behenamidopropyldiethyl amine, behenamidoethyldiethyl amine, behenamidoethyldimethyl amine, arachidamidopropyl-dimethyl amine, arachidamidopropyldiethyl amine, arachid-amidoethyldiethyl amine, arachidamidoethyldimethyl amine or mixtures thereof.

In a further embodiment, the polyglyceryl fatty ester is derived from (a) a polyglycerol component built up from 2 to 12 molecules of glycerol, based on an average, and (b) a fatty acid contains a caprylic acid, capric acid, lauric acid, myristic acid, palmitic acid, stearic acid, arachidic acid, behenic acid, lignoceric acid, cerotic acid, oleic acid, or decaoleic acid.

In another aspect, the polyglyceryl fatty ester contains one or more of a polyglyceryl-10 laurate, polyglyceryl-10 decaoleate; polyglyceryl-3 monostearate; polyglyceryl-6 distearate, polyglyceryl-10 stearate; polyglyceryl-10 oleate; polyglyceryl-10 dipalmitate, or polyglyceryl-10 caprylate/caprate

In one particular embodiment, at least one polyglyceryl ester having an HLB from about 12 to 18. One particular polyglyceryl fatty ester is polyglyceryl-10 laurate that has a HLB of about 15.

In further aspect, the present invention provides a hair cleansing composition that is essentially free of an anionic surfactant.

In one embodiment, the fatty alcohol contains a saturated, unsaturated, straight or branched chain fatty alcohol having a carbon chain length from about C₈-C₃₆ atoms.

In one particular embodiment, the fatty alcohol contains a arachidyl alcohol, behenyl alcohol, capric alcohol, capryl alcohol, ceryl alcohol, cetyl alcohol, cis4-t-butylcyclohexanol, decyl alcohol, dodecyl alcohol, erucyl alcohol, isostearyl alcohol, isocetyl alcohol, lauryl alcohol, linalool, lignoceryl alcohol, myristyl alcohol, oleyl alcohol, pelargonic alcohol, palmitoleyl alcohol, stearyl alcohol, or a undecyl alcohol.

In a further embodiment, the weight ratio of cationic surfactant to fatty alcohol is from about 1:1 to 1:10.

In yet another embodiment, the composition further contains one or more additives selected from conditioning agents, fragrances, dyes, structuring aids, fillers, pH adjustment agents, chelating agents, antidandruff agents, biocidal agent, essential oils, binding agent, natural extracts, botanical extracts, organic acids, alcohols, emulsifiers, natural plant material, essential fatty acids, preservatives, antioxidants, moisturizing agent, or coloring agent, pearlescent agent, stabilizer, humectant or foam modifier, thickening agent, or vitamins.

In one particular embodiment, the composition contains a) 0.01 w/w %-20 w/w % of at least one cationic surfactant, b) 0.01 w/w %-20 w/w % of at least one or more polyglyceryl ester, and c) 0.01 w/w %-15 w/w % of a saturated, unsaturated, straight or branched chain fatty alcohol having a carbon chain length from about C₈-C₃₆ atoms; wherein the composition is essentially free of an anionic surfactant.

In another aspect, the present invention provides a method for cleansing hair said method comprises adding an effective amount of the composition according to claim 1 to the hair, massaging the hair and rinsing the hair after a reaction time.

These and other aspects will become apparent when reading the detailed description of the invention.

### DETAILED DESCRIPTION

It has now been surprisingly found that a hair cleansing and conditioning composition can be prepared containing a cationic surfactant, a polyglyceryl ester, and at least one fatty alcohol. This hair cleansing and conditioning composition is also referred to as “cleansing conditioners”. The composition has advantages of being able to cleanse hair and the scalp, without the need for harsh anionic surfactants. In one embodiment, the composition is essentially free of anionic surfactants. The addition of polyglyceryl esters as a nonionic surfactant provides cleansing improvement without compromising conditioning properties for hair treatment. The hair cleansing and conditioning composition allows the hair treated with the composition to retain the natural oil in hair, and returns the luster, shine and health to hair. Further, the hair cleansing and conditioning formulation is gentler on dyed or otherwise colored hair; allows the hair to retain the applied color, as compared to other hair cleansing and conditioning formulation.

As used herein, the term “essentially free” herein, refers to a content of anionic (sulphate) surfactants less than 1 w/w %, more suitably of at most about 0.50 w/w %, particularly at most about 0.10 w/w %, and in most particular completely free of the anionic surfactants.

One aspect of the present disclosure provides a hair cleansing and conditioning composition containing a) 0.01 w/w %-20 w/w % of at least one cationic surfactant; b) 0.01 w/w %-20 w/w % of at least one or more nonionic surfactant; wherein at least one nonionic surfactant comprises a polyglyceryl ester; and c) 0.01 w/w %-15 w/w %, of at least one fatty alcohol. While the amounts of these ingredients do not add up to 100% other ingredients such as carriers or solvents may be present in the composition making up the 100%. Other ingredients mentioned below can also be present in the cleansing conditioner composition. For example, the present invention describes a hair cleansing composition containing a) 0.01 w/w %-20 w/w % of at least one cationic surfactant; wherein at least one cationic surfactant comprises, b) 0.01 w/w %-20 w/w % of at least one or more nonionic surfactant; wherein at least one nonionic surfactant comprises a polyglyceryl ester; c) 0.01 w/w %-15 w/w %, of at least one fatty alcohol, and d) an aqueous or non-aqueous solvent; wherein the total composition equals to 100%.

The cationic surfactant may include, but are not limited to, quaternary ammonium compounds, esterquats, amidoamines, amines with amide linkages, polyoxyethylene alkyl & alicyclic amines 4-n,n,n′,n′ tetrakis substituted ethylenediamines; 2-alkyl 1-hydroxethyl 2-imidazolines, combinations thereof, and the like. Additionally, the quaternary ammonium compound may comprise those materials having the CTFA designations Quaternium-5, Quaternium-31 or Quaternium-18; and the like. Suitably, the composition contains a quaternary ammonium compound, an amidoamine compound or a mixture thereof.

Exemplary quaternary ammonium compounds include quaternary a cetyltrimethylammonium compound, behenyltrimethylammonium compound, cetylpyridinium compound, tetramethylammonium compound, tetraethylammonium compound, octyltrimethylammonium compound, dodecyltrimethylammonium compound, hexadecyltrimethylammonium compound, octyldimethylbenzylammonium compound, decyldimethylbenzylammonium compound, stearyldimethylbenzylammonium compound, didodecyldimethylammonium compound, dioctadecyldimethylammonium compound, tallowtrimethylammonium compound, dihydrogenated tallow dimethyl ammonium compound, cocotrimethylammonium compound, PEG-2-oleammonium compound or a mixture of two or more of these compounds. Each of the compounds may have, wherein each of the above compounds has a halide or a sulfate anion. Suitable halides include, for example chloride and bromide. Suitable sulfates include, for example, methosulfate.

Typically, chloride compounds are used in personal care formulation. Examples of the chloride quaternary ammonium compounds include, for example, but are not limited to, alkyltrimethylammonium chlorides, dialkyldimethylammonium chlorides, and/or trialkylmethylammonium chlorides such as cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, dihydrogenated tallow dimethyl ammonium chloride cocotrimethylammonium chloride, PEG-2-oleammonium chloride or mixtures thereof.

Suitably, the quaternary ammonium compound cetyltrimethylammonium chloride, available commercially, for example as Carsoquat CT429, ex Lonza; behenyltrimethylammonium chloride, available commercially, for example as GENAMIN KDMP, ex Clariant; combinations thereof, are of particular interest in the composition of the present disclosure.

The hair cleansing composition may comprise at least one amidoamine. Examples of aminodamines include, but are not limited to, stearamido-propyldimethyl amine, stearamidopropyldiethyl amine, stearamidoethyldiethyl amine, stearamidoethyldimethyl amine, palmitamidopropyldimethyl amine, palmitamidopropyl-diethyl amine, palmitamidoethyldiethyl amine, palmitamidoethyldimethyl amine, behenamidopropyldimethyl amine, behenamidopropyldiethyl amine, behenamidoethyldiethyl amine, behenamidoethyldimethyl amine, arachidamidopropyl-dimethyl amine, arachidamidopropyldiethyl amine, arachid-amidoethyldiethyl amine, arachidamidoethyldimethyl amine, or mixtures thereof, and the like.

Suitable examples of aminodamines include stearamidopropyldimethyl amine, stearamidoethyldiethyl amine, mixtures thereof, and the like.

Suitable esterquats include, but are not limited to, quaternized ester salts of fatty acids with triethanolamine, quaternized ester salts of fatty acids with diethanolalkylamines, and quaternized ester salts of fatty acids with 1,2-dihydroxypropyldialkylamines. Examples of ester quats include, but are not limited to, the trademarks Stepantex®, Dehyquart® and Armocare®. The products Armocare® VGH-70, an N,N-bis(2-palmitoyloxyethyl)dimethylammonium chloride, and Dehyquart® F-75, Dehyquart® C-4046, Dehyquart® L80 and Dehyquart® AU-35 are examples of such esterquats.

Typically, the cationic surfactant may be present in an amount between 0.01 w/w %-20 w/w %; suitably in an amount between 0.01 w/w %-15 w/w %; and desirably more suitably in an amount between 0.1 w/w %-10 w/w %; based on the weight of the total composition.

The cleansing and conditioning composition contains a polyglyceryl fatty ester. The polyglyceryl fatty ester in the hair cleansing and conditioning composition is typically present in an amount by weight of from about 0.01 w/w % to about 20 w/w %, more typically from about 0.10 w/w/% to about 10 w/w %, particularly from about 0.50 w/w % to about 7.00 w/w %, based on the total weight of the hair cleansing and conditioning composition.

Polyglyceryl esters useable in the present invention may be formed from saturated, unsaturated, natural or synthetic fatty acids, and the like. For instance, saturated fatty acids include, but are not limited to, caprylic acid, capric acid, lauric acid, myrystic acid, palmitic acid, stearic acid, arachidic acid, behenic acid, lignoceric acid, cerotic acid, oleic acid, decaoleic acid, combinations thereof, derivatives thereof, and the like.

Furthermore, the polyglyceryl esters are derived from (a) a polyglycerol component built up from 2 to 12 molecules of glycerol, based on an average, and (b) a fatty acid selected from the group consisting of caprylic acid, capric acid, lauric acid, myristic acid, palmitic acid, stearic acid, arachidic acid, behenic acid, lignoceric acid, cerotic acid, oleic acid, decaoleic acid, mixtures thereof, and the like.

Examples of polyglyceryl ester, having an HLB value between 1-9 or 10-16, includes, but are not limited to, polyglyceryl monodecaoleate such as polyglyceryl-10 decaoleate; polyglyceryl monooleate such as polyglyceryl-2-monooleate, polyglyceryl-3 monooleate, polyglyceryl-4 monooleate, polyglyceryl-6 monooleate, or polyglyceryl-10 monooleate; polyglyceryl dioleate such as polyglyceryl-2 dioleate, polyglyceryl-3 dioleate, polyglyceryl-5 dioleate, polyglyceryl-6 dioleate or polyglyceryl-10 dioleate; polyglyceryl trioleate such as polyglyceryl-5 trioleate or polyglyceryl-10 trioleate; polyglyceryl tetraoleate such as polyglyceryl-2 tetraoleate, polyglyceryl-6 tetraoleate, or polyglyceryl-10 tetraoleate; polyglyceryl pentaoleate such as polyglyceryl-4 pentaoleate, polyglyceryl-6 pentaoleate, or polyglyceryl-10 pentaoleate; polyglyceryl heptaoleate such as polyglyceryl-6 heptaoleate, polyglyceryl-10 heptaoleate; polyglyceryl monostearate such as polyglyceryl-2 monostearate, polyglyceryl-3 monostearate, polyglyceryl-4 monostearate, polyglyceryl-5 monostearate, polyglyceryl-6 monostearate or polyglyceryl-10 monostearate; polyglyceryl distearate such as polyglyceryl-2 distearate, polyglyceryl-3 distearate, polyglyceryl-4 distearate, polyglyceryl-6 distearate, or polyglyceryl-10 distearate; polyglyceryl tristearate such as polyglyceryl-4 tristearate, polyglyceryl-5 tristearate, polyglyceryl-6 tristearate, or polyglyceryl-10 tristearate; polyglyceryl tetrastearate such as polyglyceryl-2 tetrastearate; polyglyceryl pentastearate such as polyglyceryl-4 pentastearate, polyglyceryl-6 pentastearate, or polyglyceryl-10 pentastearate; polyglyceryl heptastearate such as polyglyceryl-10 heptastearate; polyglyceryl isostearate such as polyglyceryl-2 isostearate, polyglyceryl-3 isostearate, polyglyceryl-4 isostearate, polyglyceryl-6 isostearate, or polyglyceryl-10 isostearate; polyglyceryl diisostearate such as polyglyceryl-2 diisostearate, polyglyceryl-3 diisostearate, polyglyceryl-4 diisostearate, polyglyceryl-6 diisostearate, polyglyceryl-10 diisostearate, or polyglyceryl-15 diisostearate; polyglyceryl triisostearate such as polyglyceryl-2 triisostearate, polyglyceryl-3 triisostearate, polyglyceryl-5 triisostearate, polyglyceryl-10 triisostearate; polyglyceryl tetraisostearate such as polyglyceryl-2 tetraisostearate; polyglyceryl caprylate such as polyglyceryl-2 caprylate, polyglyceryl-3 caprylate, polyglyceryl-4 caprylate, polyglyceryl-6 caprylate, or polyglyceryl-10 caprylate; polyglyceryl dicaprylate such as polyglyceryl-5 dicaprylate; polyglyceryl sesquicaprylate such as polyglyceryl-2 sesquicapyrlate; polyglyceryl octacaprylate such as polyglyceryl-6 octacaprylate; polyglyceryl caprate such as polyglyceryl-2 caprate, polyglyceryl-3 caprate, polyglyceryl-4 caprate, polyglyceryl-5 caprate, polyglyceryl-6 caprate, polyglyceryl-10 caprate: polyglyceryl dicaprate such as polyglyceryl-3 dicaprate or polyglyceryl-6 dicaprate; polyglyceryl caprylate/caprate such as polyglyceryl-4 capyrlateicaprate, polyglyceryl-6 caprylate/caprate, or polyglyceryl-10 caprylate/caprate; polyglyceryl palmitate such as polyglyceryl-2 palmitate, polyglyceryl-3 palmitate, polyglyceryl-6 palmitate or polyglyceryl-10 palmitate; polyglyceryl dipalmitate such as polyglyceryl-6 dipalmitate or polyglyceryl-10 dipalmitate; polyglyceryl tetrabehenate such as polyglyceryl-6 tetrabehenate; polyglyceryl myristate such as polyglyceryl-6 myristate or polyglyceryl-10 myristate; polyglyceryl rincinoleate such polyglyceryl-6 polyricinoleate or polyglyceryl-10 ricinoleate; other derivatives thereof, or mixtures thereof, and the like.

A variety of polyglyceryl esters in various forms may be used, specifically a polyglyceryl-10 laurate, polyglyceryl-10 decaoleate; polyglyceryl-3 monostearate; polyglyceryl-6 distearate, polyglyceryl-10 stearate; polyglyceryl-10 oleate; polyglyceryl-10 dipalmitate, polyglyceryl-10 caprylate/caprate, or mixture thereof Typically, the cleansing and conditioning composition advantageously contains a polyglyceryl-10 laurate as the polyglyceryl fatty ester component. Typically, the composition comprises at least one polyglyceryl ester having an HLB from about 12 to 18.

In addition to the polyglyceryl ester, other nonionic surfactants may be used in combination with the polyglyceryl ester. Examples of suitable non-ionic surfactants include, but are not limited to, alkyl(oligo)glycosides, fatty acid alkanolamides, amine oxides, alkyl polyglucosides, sorbitan esters, ethoxylated esters, polyethyelene glycol esters, polyoxyethylene fatty acid amides, ethoxylated derivatives, polyoxyethylene surfactants, polyoxyethylene sorbitan esters, combinations thereof, and the like.

Examples of alkyl(oligo)glycoside, include but are not limited to, caprylyl/capryl glucoside, decyl glucoside, lauryl glucoside and/or coco glucoside, mixtures thereof, and the like.

The proportion of the non-ionic surfactant, including the polyglyceryl ester, will typically in a proportion by weight of from about 0.01 w/w % to about 20 w/w %, more suitably from about 0.10 ww/% to about 10 w/w %, particularly desirably from about 0.50 w/w % to about 7.00 w/w %, based on the total weight of the hair cleansing conditions. For example, an additional non-ionic surfactants such as amine oxides may be used in combination with the polyglyceryl ester surfactant. Examples of amine oxides include, but are not limited to, stearamido-propyldimethylamine, stearamidopropyldiethylamine, stearamidoethyldiethylamine, stearamidoethyldimethylamine, palmitamidopropyldimethylamine, palmitamidopropyl-diethylamine, palmitamidoethyldiethylamine, palmitamidoethyldimethylamine, behenamidopropyldimethyl-amine, behenamidopropyldiethylmine, behenamidoethyldiethyl-amine, behenamidoethyldimethylamine, arachidamidopropyl-dimethylamine, arachidamidopropyldiethylamine, arachid-amidoethyldiethylamine, arachidamidoethyldimethylamine or combinations thereof, and the like.

Suitable examples of amine oxide include, stearamidopropyldimethylamine with tradenames LEXAMINE S-13 available from Inolex (Philadelphia, Pa., USA) and AMIDOAMINE MSP available from Nikko (Tokyo, Japan); stearamidoethyldiethylamine with a tradename AMIDOAMINE S available from Nikko; behenamidopropyldimethylamine with a tradename INCROMINE BB available from Croda (North Humberside, England); and various amidoamines with tradenames SCHERCODINE series available from Scher (Clifton, N.J., USA), or mixtures thereof, and the like.

In addition to the cationic surfactant and the polyglyceryl ester compounds and other nonionic surfactants (when present), the hair cleansing and conditioning composition further contains a fatty alcohol which is a saturated, unsaturated, straight or branched chain fatty alcohol having a carbon chain length from about C₈-C₃₆ carbon atoms. Typically, the fatty alcohol comprises a carbon chain length from about C₁₀-C₂₂ carbon atoms; or desirably a carbon chain length from about C₁₄-C₂₂ carbon atoms.

Exemplary examples of fatty alcohols include, but are not limited to, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linoyl alcohol, linolenyl alcohol, elaeostearyl alcohol, arachyl alcohol, gadoleyl alcohol, behenyl alcohol, erucyl alcohol and brassidyl alcohol, or mixtures thereof, and the like. The water-soluble alcohols are preferred, such as ethanol, 1,2-propylene glycol, glycerol, sorbitol, benzyl alcohol and/or phenoxyethanol or mixtures thereof and the like.

Suitably, the fatty alcohol is a fatty alcohol selected from arachidyl alcohol, behenyl alcohol, capric alcohol, capryl alcohol, ceryl alcohol, cetyl alcohol, cis4-t-butylcyclohexanol, decyl alcohol, dodecyl alcohol, erucyl alcohol, isostearyl alcohol, isocetyl alcohol, lauryl alcohol, linalool, lignoceryl alcohol, myristyl alcohol, oleyl alcohol, pelargonic alcohol, palmitoleyl alcohol, stearyl alcohol, undecyl alcohol, or combinations thereof.

The hair cleansing and conditioning composition will have the fatty alcohol present in an amount between 0.01 w/w %-15 w/w %, based on the total weight of the composition; suitably in an amount between 0.1 w/w %-10 w/w %; or desirably in an amount between 0.25 w/w %-7 w/w %; based on the total weight of the composition.

Further, the hair cleansing and conditioning composition may contain the cationic surfactant and a fatty alcohol present in weight ratio of cationic surfactant to fatty alcohol is in the range of from 1:1 to 1:10; typically the weight ratio of cationic surfactant to fatty alcohol is from 1:1.5 to 1:8, and more typically from 1:2 to 1:5.

As is mentioned above, the hair cleansing and conditioning composition may contain a solvent or a carrier for the components in the composition. One example of solvents or carriers includes aqueous solvents, which include, but are not limited to, water, aqueous alcohols, ammonia water, acid solutions, salt solutions, water-miscible organic solvents, polyhydroxy alcohols (such as glycerin, diglycerin, propylene glycol, dipropylene glycol, sorbitol, pantenol and sugar); urea, alpha-hydroxy acid and its salt; and low molecular weight polyethylene glycols with molecular weight less than 20,000; water soluble or dispersible polymers; or mixtures thereof; and the like.

The term “aqueous alcohol” herein, refers to saturated, unsaturated, straight or branched hydrocarbon chain having C₂-C₄ carbon atoms and at least one hydroxyl groups, wherein the hydrocarbon chain may optionally comprise one or several heteroatoms (such as oxygen or sulfur).

Examples of non-aqueous solvents include, but are not limited to, non-aqueous alcohols, univalent or polyvalent alcohols, alkanolamines, or glycol ethers, any substance immiscible in water, mixtures thereof, and the like.

Alternately, in another aspect of the present composition is the hair cleansing and conditioning composition may further contain 0.1 w/w %-20 w/w % of additional surfactants, such as an amphoteric surfactants, amphoteric/zwitterionic or mixtures thereof. Generally, the hair cleansing and conditioning composition is free of harsh anionic surfactants, as described above.

Examples of exemplary amphoteric/zwitterionic surfactants include, but are not limited to, alkyl amphoacetates, alkyl amphodiacetates, sultaines, amphosulfonates, betaines, alkyl amphopropionates, alkyl amphodipropionates or mixtures thereof, and the like.

Suitable amphoteric surfactants include, but are not limited to, sodium cocoamphoacetate, disodium cocoamphodiacetate, sodium lauroamphoacetate, sodium lauroamphodiacetate, sodium cocoamphopropionate, disodium cocoamphodipropionate, cocobetaine, laurylbetaine, cocoamidopropylbetaine, lauramidopropylbetaine, mixtures thereof, and the like

A variety of additional ingredients may be included in the hair cleansing and conditioning composition. These additional ingredients include, for example, conditioning agents, fragrances, dyes, structuring aids, fillers, pH adjustment agents, chelating agents, antidandruff agents, biocidal agent, essential oils, binding agent, natural extracts, botanical extracts, organic acids, alcohols, emulsifiers, natural plant material, essential fatty acids, preservatives, antioxidants, moisturizing agent, or coloring agent, pearlescent agent, stabilizer, humectant or foam modifier, thickening agent, or vitamins, and the like. Typically, any variation of additional ingredients may be present in the hair cleansing composition in an amount up to about 50 w/w %.

Examples of antidandruff agents include, but are not limited to, spiculisporic acid and or salts thereof; essential oils, natural extracts, zinc pyrithione, piroctone olamine or selenium disulfide, acetylsalicylic acid, hydroxypyridinone or derivatives thereof [such as 2(1H)-Pyridinone, 1-hydroxy-6-(octyloxy)-; 2(1H)-Pyridinone, 1-hydroxy-6-(octyloxy)-, compd. with 2-aminoethanol (1:1)]; hydroxypyrimidinone derivatives, octopirox (piroctone olamine), pyrithione derivatives or salts thereof (such as calcium, magnesium, strontium, barium, zinc, cadmium, tin and zirconium salts;); bispyrithione, octenidine or salts thereof; azole compounds (such as climbazole, ketoconazole, clotrimazole, econazole, isoconazole and miconazole), selenium or salts thereof, salicylic acid or salts thereof, or tea tree oil; antifungal polymers (such as amphotericin B or nystatin; ellagic acid, ethers thereof, ellagic acid salts and salts of its ethers (such as alkali metal or alkaline-earth metal salts, especially the sodium, potassium, calcium or magnesium salts)); sulfur in its various forms, cadmium sulfide, allantoin, coal or wood tars and derivatives thereof; undecylenic acid, fumaric acid, and allylamines such as terbinafme; antifungal peptides (such as syringomycins, syringostatins, syringotoxins, nikkomycins, echinocandins, pneumocadins, aculeacins, mulundocadins, cecropins, α-defensins, β-defensins, novispirins, and mixtures thereof); or combinations thereof, and the like.

In one aspect of the present invention, the hair cleansing and conditioning composition contains an antidandruff agent further comprises piroctone olamine, zinc pyrithione, salicylic acid, climbazole, coal tar, selenium sulphide or hydroxypyridinone or derivatives thereof.

Examples of preservatives include, but are not limited to, benzoic acid or salts thereof, benzyl alcohol, sorbic acid or salts thereof, parabens, dehydroacetic acid, bronopol, triclosan, imidazolidinyl urea, Polyaminopropyl Biguanide (PHMB), phenoxyethanol, hydantoins (i.e. DMDMH), isothiazolones, chlorhexidine, diazolidinyl urea, chlorphenesin, sodium hydroxymethylglycinate, benzethonoium chloride, ethylhexylglycerol, IPBC (iodopropynyl butylcarbamate), salicylic acid or salts thereof, potassium salts, natural acids, and the like.

For example, if desired, the hair cleansing and conditioning composition may contain a) 0.001 w/w %-20 w/w % of at least one cationic surfactant, b) 0.01 w/w %-20 w/w % of at least one or more polyglyceryl ester, c) 0.01 w/w %-15 w/w % of a saturated, unsaturated, straight or branched chain fatty alcohol having a carbon chain length from about C₈-C₃₆ atoms; d) a solvent or carrier and e) 0.01 w/w %-15 w/w % of at least one antidandruff agent. In addition the hair cleansing and conditioning composition is essentially free of an anionic surfactant, typically completely free of an anionic surfactant.

Typically, the hair cleansing composition may comprise a) 0.001 w/w %-20 w/w % of at least one cationic surfactant, b) 0.01 w/w %-20 w/w % of at least one or more polyglyceryl ester, and c) 0.01 w/w %-15 w/w % of a saturated, unsaturated, straight or branched chain fatty alcohol having a carbon chain length from about C₈-C₃₆ atoms; wherein the composition is essentially free of an anionic surfactant.

Suitably, the hair cleansing composition may comprise aa) 0.001 w/w %-15 w/w % of at least one cationic surfactant; b) 0.01 w/w %-10 w/w % of at least one or more polyglyceryl ester, and c) 0.01 w/w %-10 w/w % of a saturated, unsaturated, straight or branched chain fatty alcohol having a carbon chain length from about C₈-C₂₈ atoms; wherein the composition is essentially free of an anionic surfactant.

In another aspect, the present invention provides a method for cleansing and conditioning hair. The method includes adding an effective amount of the hair cleansing and conditioning composition, to the hair. Followed by massaging the hair with the composition and rinsing the hair after a period of time. Typically, the messaging time is between 5 seconds and 5 minutes, and the rinsing is done for a period of time to remove excess composition from the hair.

The hair cleansing and conditioning composition is useful for mild cleansing, effectively removing dirt and sebum from the hair while providing a highly effective care of hair and scalp for damaged, entangled or dull hair. The hair cleansing conditioners according to exemplary embodiments herein are conventional rinse-off products, such as a nourishing shampoo and/or a foaming, rinse-off nourishing conditioner.

In other embodiments, the hair cleansing conditioners as contemplated herein are applied as cleansing hair foams. This guarantees a particularly good distribution and dosability of the hair cleansing conditioners. A particularly good distribution and dosing of the hair cleansing conditioners is ensured as a result. The hair cleansing conditioners as contemplated herein have an improved hair compatibility compared to a conventional nourishing shampoo based on anionic sulphate surfactants.

Other advantages of hair cleansing and conditioning composition is the formulation is gentle on dyed or colored hair. It has been discovered that the hair cleansing conditioner is less likely to remove hair coloring from the dyed hair.

The following examples illustrate the invention without limitation. All parts and percentages are given by weight unless otherwise indicated.

It will be understood that each of the elements described in the examples below, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims.

### Examples 1

A cleansing conditioner composition was prepared by mixing the ingredients shown in Table 1. All percentages are weight percent.

TABLE 1

| | | | Example |
|---|---|---|---|
| | | Control CD | 1 |
| | | As is | As is |
| Chemical/Trade name | Active % | % | % |
| cetyltrimethylammonium chloride | 29 | 4.62 | 4.62 |
| Stearyl alcohol | 100 | 1.2 | 1.2 |
| Cetyl Alcohol | 100 | 2.8 | 2.8 |
| polyglyceryl-10 laurate | 100 | | 1 |
| Disodium EDTA | 100 | 0.1 | 0.1 |
| Cosmacil CQ preservative | 55 | 0.5 | 0.5 |
| (Polyaminopropyl Biguanide) | | | |
| Glycacil 2000 preservative | | 0.075 | 0.075 |
| (lodopropynyl Butylcarbamate) | | | |
| KCl (potassium chloride) | 100 | 0.15 | 0.15 |
| Aqua | 100 | 90.555 | 89.555 |

The formulations were tested on 2.5 g human hair switches. The switches were treated with 1 g of a 5% sebum solution applied to each switch of hair to simulate dirty on the hair. Each switch of hair was hair was combed and allowed to dry overnight. The next day the dirty switches of hair were treateate with 0.5 g of the compositions shown in Table 1 Each hair switch was washed for 30 seconds, the rinsed with water for 30 seconds. The water was applied to the hair at 38 C at a rate of 4 liters per minute.

A panel of 12 panelist were caused to make 6 choices of hair switches with respect to which hair switch looked visually clearer than the others. All panelist selected the hair switches treated with the composition Example 1 as looking the cleanest. Next the panelist were asked to feel the hair and determine which hair felt the cleanest. 71 out of 72 selected the hair treated with Example 1 as feeling cleaner that the hair treated with the control composition. This is statistically significant with a p<0.001. The hair treated with the composition of Example 1 appeared to be visually cleaner and felt tactically cleaner that the control.

### Example 2

The same compositions shown in Table 1 were used to treat hair tresses. The treated hair tresses were placed in a Diastron MTT175 with combing attachments measures the ease of combing in the wet. The comb is driven through a wet hair tress whilst the load cell measures the force required to do so. This information allows us to calculate the work done to comb through a wet hair tress. The composition of Example 1 and the control formulation both had a significant reduction in the work done wet-combing when compared to the base-wash control and perform parity to the Control Conditioner shown in Table 1. The formulation containing the polyglyceryl ester combed just as easy as the control example, shown that the polyglyceryl ester does not reduce the wet combability of hair treated with Example 1.

### Example 3

The compositions of Table 2 were prepared by mixing the components shown in the Table. The testing of Example 1 was repeated without a panel but just with observations of the treated switches.

TABLE 2

| | Example 3-c1 | Example 3 | Example 3-c2 | Example 3c-3 |
|---|---|---|---|---|
| | As is | As is | As is | As is |
| Chemical/Trade name | % | % | % | % |
| cetyltrimethylammonium chloride | 4.62 | 4.62 | 4.62 | 4.62 |
| Stearyl alcohol | 1.2 | 1.2 | 1.2 | 1.2 |
| Cetyl Alcohol | 2.8 | 2.8 | 2.8 | 2.8 |
| polyglyceryl-10 laurate | | 1 | | |
| cocoamine oxide | | | 1 | |
| Polysorbate 20 | | | | 1 |
| Disodium EDTA | 0.1 | 0.1 | 0.1 | 0.1 |
| Cosmacil CQ preservative | 0.5 | 0.5 | 0.5 | 0.5 |
| (Polyaminopropyl Biguanide) | | | | |
| Glycacil 2000 preservative | 0.075 | 0.075 | 0.075 | 0.075 |
| (lodopropynyl Butylcarbamate) | | | | |
| KCl (potassium chloride) | 0.15 | 0.15 | 0.15 | 0.15 |
| Aqua | 90.555 | 89.555 | 89.555 | 89.555 |

The Example 3-C1 - Control conditioner base, showed only a Conditioning benefit Example 3- - Composition according to invention exhibited both a Cleansing and Conditioning benefit
Example 3-C2 - Base + cocoamine oxide showed a Conditioning benefit, but no cleansing benefit
Example 3 - Base + Polysorbate 20, Cleansing benefit with loss of conditioning

The above example shows the unique combination of properties obtained using the combination of the polyglyceryl ester with the cationic surfactant. The same results are not obtained with other non-ionic surfactants.

### Example 4

The composition of Example 1 is compared to a commercially available low shampoo gentle cleansing cream containing Aqua, cetearyl alcohol, behentrimonium chloride, coco-betaine, potato starch modified, guar hydroxypropyltrimonium chloride, tocopherol, Cocos nucifera oil (coconut), sodium chloride, sodium benzoate, phenoxyr=ethanol, ethylhexyl methoxycinnamate, trideceth-6, polyquaternium-7, polylinseed oil, fumaric acid, benzyl alcohol, benzyl salicylate, linalool, benzophenone-4, amodimethicone, isopropyl alcohol, alpha-isomethylionone, geraniol, cetyl hydroxyethylcellulose, butylphenyl methylpropional, Citronellol, cetrimonium chloride, citric acid, BHT, hextl cinnamal, glycerine, parfum.

The testing of example 1 was repeated. The Panelist selected Example 1 as having the cleanest feel in 45 of 58 selections.

### Example 5

The formulation of Example 1 was compared to the commercial formulation of available from L'Oreal, sold as “Color Protect Shampoo” and “Color Protect Cleansing conditioner on dyed hair. Hair switches were bleached and coloured with 66/46 Vibrant red dye. Hair Colour (L*, a* and b*) was measured over several washes, same method of Example 1 to determine if any treatments reduce the overall change in the hair colour over time (ΔE), they may offer a colour protection benefit. The results show the Cleansing Conditioner containing 1% 10-1-L Polyaldo provides significantly better colour protection when compared to the L'Oreal Colour Protect Shampoo after 5 and 7 washes. The results are shown in Table 3. The composition of Example 1 is significantly better than the commercial products after 3, 5 and 7 washes.

TABLE 3

| | | | After | After |
|---|---|---|---|---|
| ΔE | After 1 wash | After 3 washes | 5 washes | 7 washes |
| Color Protect ™ | 2.55 | 5.03 | 77.6 | 10.16 |
| Shampoo | | | | |
| Example 1 | 2.06 | 4.12 | 5.49 | 6.91 |
| composition | | | | |
| Color Protect ™ | 2.78 | 5.47 | 7.91 | 9.95 |
| Cleansing | | | | |
| Conditioner | | | | |

While the invention has been described above with references to specific embodiments thereof, it is apparent that many changes, modifications and variations can be made without departing from the invention concept disclosed herein. Accordingly, it is intended to embrace all such changes, modifications, and variations that fall within the spirit and broad scope of the appended claims.

What is claimed is:

1. A hair cleansing and conditioning composition comprising
a) 0.001 w/w %-20 w/w % of a cationic surfactant;
b) 0.01 w/w %-20 w/w % of a polyglyceryl ester surfactant; and
c) 0.01 w/w %-15 w/w %, of at least one fatty alcohol.

2. The hair cleansing and conditioning composition, according to claim 1, wherein the cationic surfactant comprises quaternary ammonium compound, an amidoamine compound or a mixture thereof.

3. The hair cleansing and conditioning composition according to claim 2, wherein the quaternary ammonium compound comprises cetyltrimethylammonium compound, a behenyltrimethylammonium compound, cetylpyridinium compound, tetramethylammonium compound, tetraethylammonium compound, octyltrimethylammonium compound, dodecyltrimethylammonium compound, hexadecyltrimethylammonium compound, octyldimethylbenzylammonium compound, decyldimethylbenzylammonium compound, stearyldimethylbenzylammonium compound, didodecyldimethylammonium compound, dioctadecyldimethylammonium compound, tallowtrimethylammonium compound, dihydrogenated tallow dimethyl ammonium compound, cocotrimethylammonium compound, PEG-2-oleammonium chloride or a mixture of two or more of these compounds, wherein each of the above compounds has a halide or a sulfate anion.

4. The hair cleansing and conditioning composition according to claim 2, wherein the amidoamine compound comprises a stearamido-propyldimethyl amine, stearamidopropyldiethyl amine, stearamidoethyldiethyl amine, stearamidoethyldimethyl amine, palmitamidopropyldimethyl amine, palmitamidopropyl-diethyl amine, palmitamidoethyldiethyl amine, palmitamidoethyldimethyl amine, behenamidopropyldimethyl amine, behenamidopropyldiethyl amine, behenamidoethyldiethyl amine, behenamidoethyldimethyl amine, arachidamidopropyl-dimethyl amine, arachidamidopropyldiethyl amine, arachid-amidoethyldiethyl amine, arachidamidoethyldimethyl amine, or mixtures thereof.

5. The hair cleansing and conditioning composition according to claim 1, wherein the polyglyceryl fatty ester is derived from (a) a polyglycerol component built up from 2 to 12 molecules of glycerol, based on an average, and (b) a fatty acid comprises a caprylic acid, capric acid, lauric acid, myristic acid, palmitic acid, stearic acid, arachidic acid, behenic acid, lignoceric acid, cerotic acid, oleic acid, decaoleic acid or a mixture thereof.

6. The hair cleansing and conditioning composition according to claim 5, wherein the polyglyceryl fatty ester comprises polyglyceryl-10 laurate, polyglyceryl-10 decaoleate; polyglyceryl-3 monostearate; polyglyceryl-6 distearate, polyglyceryl-10 stearate; polyglyceryl-10 oleate; polyglyceryl-10 dipalmitate, polyglyceryl-10 caprylate/caprate or a mixture thereof.

7. The hair cleansing and conditioning composition according to claim 1, wherein the polyglyceryl ester has an HLB from about 12 to about 18.

8. The hair cleansing and conditioning composition according to claim 6, wherein the polyglyceryl esters is present in an amount between 0.01 w/w %-10 w/w %, based on the total weight of the composition.

9. The hair cleansing and conditioning composition according to claim 1, wherein the composition is essentially free of an anionic surfactant.

10. The hair cleansing and conditioning composition according to claim 1, wherein the fatty alcohol comprises a saturated, unsaturated, straight or branched chain fatty alcohol having a carbon chain length from about C₈-C₃₆ carbon atoms.

11. The hair cleansing and conditioning composition according to claim 10, wherein the fatty alcohol comprises a arachidyl alcohol, behenyl alcohol, capric alcohol, capryl alcohol, ceryl alcohol, cetyl alcohol, cis4-t-butylcyclohexanol, decyl alcohol, dodecyl alcohol, erucyl alcohol, isostearyl alcohol, isocetyl alcohol, lauryl alcohol, linalool, lignoceryl alcohol, myristyl alcohol, oleyl alcohol, pelargonic alcohol, palmitoleyl alcohol, stearyl alcohol, or a undecyl alcohol.

12. The hair cleansing and conditioning composition according to claim 1, wherein the weight ratio of cationic surfactant to fatty alcohol is from about 1:1 to about 1:10.

13. The hair cleansing and conditioning composition according to claim 1, further comprising 0.1 w/w %-20 w/w % of at least or more surfactants; wherein at least one or more comprises an amphoteric surfactants or amine oxide.

14. The hair cleansing and conditioning composition according to claim 1, further comprising an additive selected from conditioning agents, fragrances, dyes, structuring aids, fillers, pH adjustment agents, chelating agents, antidandruff agents, biocidal agent, essential oils, binding agent, natural extracts, botanical extracts, organic acids, alcohols, emulsifiers, natural plant material, essential fatty acids, preservatives, antioxidants, moisturizing agent, or coloring agent, pearlescent agent, stabilizer, humectant, foam modifier, thickening agent, vitamins or mixtures thereof.

15. The hair cleansing and conditioning composition according to claim 1, further comprising an antidandruff agent selected from piroctone olamine, zinc pyrithione, salicylic acid, climbazole, coal tar, selenium sulphide or a mixture thereof.

16. The hair cleansing and conditioning composition according to claim 1, comprising a) 0.001 w/w %-20 w/w % of a cationic surfactant, b) 0.01 w/w %-20 w/w % of a polyglyceryl ester, and c) 0.01 w/w %-15 w/w % of a saturated, unsaturated, straight or branched chain fatty alcohol having a carbon chain length from about C₈-to about C₃₆ atoms; and wherein the composition is essentially free of an anionic surfactant.

17. The hair cleansing and conditioning composition according to claim 16, comprising a) 0.001 w/w %-15 w/w % of a cationic surfactant; b) 0.01 w/w %-10 w/w % of a polyglyceryl-10 laurate, and c) 0.01 w/w %-10 w/w % of a saturated, unsaturated, straight or branched chain fatty alcohol having a carbon chain length from about C₁₀-C₂₈ atoms; wherein the composition is essentially free of an anionic surfactant.

18. The hair cleansing and conditioning composition according to claim 16, comprising a) 0.001 w/w %-15 w/w % of a cetyltrimethylammonium chloride; b) 0.01 w/w %-10 w/w % of a polyglyceryl ester, and c) 0.01 w/w %-10 w/w % of a saturated, unsaturated, straight or branched chain fatty alcohol having a carbon chain length from about C14-C22 atoms; wherein the composition is essentially free of an anionic surfactant.

19. A method for cleansing and conditioning hair, said method comprises applying an effective amount of the composition according to claim 1 to the hair, massaging the composition in the hair, and rinsing the hair after a period of time.

20. The method for cleansing and conditioning hair according to claim 19, wherein the composition comprises a) 0.001 w/w %-20 w/w % of a cationic surfactant, b) 0.01 w/w %-20 w/w % of a polyglyceryl ester, and c) 0.01 w/w %-15 w/w % of a saturated, unsaturated, straight or branched chain fatty alcohol having a carbon chain length from about C8 to about C36 atoms; and wherein the composition is essentially free of an anionic surfactant.
